(19) United States
(12) Patent Application Publication
(10) Pub. No.: US 2026/0096959 A1
(43) Pub. Date: Apr. 9, 2026

(54) Method for Producing Silver Nanoparticles in an Active Carious Lesion

(71) Applicant: R.M CREIGHTON DENTAL PTY LTD, Clontarf (AU)

(72) Inventor: Graham G. Craig, Balgowlah (AU)

(73) Assignee: R.M CREIGHTON DENTAL PTY LTD, Clontarf (AU)

(21) Appl. No.: 19/345,469

(22) Filed: Sep. 30, 2025

Related U.S. Application Data
(60) Provisional application No. 63/702,677, filed on Oct. 3, 2024.

(51) Int. Cl. A61K 8/19; A61K 8/34; A61Q 11/00

(52) U.S. Cl. CPC A61K 8/19; A61K 8/345; A61Q 11/00; A61K 2800/413; A61K 2800/92

(57) ABSTRACT

A method of treating a carious lesion in a tooth which includes a step of applying a first dental solution to the carious lesion of the tooth, applying a second dental solution to the treated carious lesion within less than 10 minutes of the step of applying the first dental solution. The treated carious lesion consolidates forming a crust having a matte black surface and comprising nanoparticles after about 3-5 weeks. The first dental solution comprises an organic or inorganic silver salt and the second dental solution comprises an organic or inorganic reducing agent.

### FIELD OF THE INVENTION

The present invention relates to a tooth surface treatment that uses a first dental solution comprising an organic or inorganic silver salt, such as silver fluoride, which is applied to a carious tooth surface, followed by an application of a second dental solution comprising an organic or inorganic reducing agent such as stannous fluoride. Part of the function of the reducing agent is to synthesize silver nanoparticles (AgNPs) in vivo at body temperature.

### BACKGROUND OF THE INVENTION

A two-step topical application procedure for active carious lesions in primary molars involving the use of silver fluoride followed by stannous fluoride immediately turns a lesion black. Retention of the black stain is indicative that the underlying lesion has not progressed, whereas loss of the stain is a sign of lesion progression (Craig et al., 2013). This aspect is useful for diagnostic purposes, particularly in dental outreach programs.

During the COVID-19 pandemic when non aerosol-producing techniques were indicated, one dental facility used the above approach as an initial step for carious primary molars. This was followed by the placement of a glass-ionomer cement restoration. In essence, it followed the delayed restoration placement protocol with the Silver Modified Atraumatic Restorative Technique, which uses silver diamine fluoride (SDF) as the initial treatment (Natarajan, 2022). However, the technique described was not a recognized one because the SDF was applied with a scrubbing motion and the solution was not allowed to diffuse into the lesion. This scrubbing motion has the potential disadvantage of rubbing off the layer containing the bacteria responsible for an active carious lesion thereby negating one of the advantages of the technique.

Mei et al. (Mei 2014) describes a study which analyzed primary carious teeth treated every 6 months with silver diamine fluoride (SDF). The study compared the physico-chemical structural differences between the black layer formed in the active carious lesion with the layer formed in the arrested inactive carious lesion. The layer formed with SDF on the arrested inactive carious lesions showed a highly remineralized zone rich in calcium and phosphate having characteristics of hydroxyapatite. Needle-shaped crystallites could be seen under transmission electron microscope (TEM) in the fine particles collected from the surface of the arrested dentinal lesion. Some nanoparticles were confirmed to be silver by energy-dispersive X-ray spectrometry (EDX). However, when Mei et al. analyzed the layer formed in the active dentinal lesion, only rounded crystallites were found. Selected area electron diffraction (SAED) of these structures were devoid of arc-shaped patterns, suggesting a more random crystallite arrangement. Mei et al. did not report finding any silver nanoparticles in the layer formed in the active dentinal lesion. It is important to note that the Mei et al. paper deals with an arrested carious lesion not a lesion in the process of going from an active one to an arrested one as is the case with the instant invention.

Since there was little known of the SDF treated active carious lesions, this was worthy of further examination.

### SUMMARY OF INVENTION

To investigate the above-described situation further, a retrieved discarded crust formed with silver fluoride followed by stannous fluoride (AgF/SnF₂) treatment of active carious lesions from one patient was submitted for preliminary analysis. This investigation differs from that of Mei et al. (Mei 2014) discussed above, in that the crust was removed within about 3-5 weeks after AgF/SnF₂treatment, whereas the layer investigated by Mei et al. was removed 6 months after SDF treatment. It was found that when the gap between initial treatment and restoration placement was within 3-5 weeks, a black, friable crust containing nanoparticles comprising silver was seen on the caries'surface.

In an aspect, the invention is drawn to a method of treating an active carious lesion in a tooth comprising steps of: applying a first dental solution to the carious lesion of the tooth, applying a second dental solution to the treated carious lesion within less than 10 minutes of the step of applying the first dental solution, wherein the treated carious lesion consolidates forming a crust having a matte black surface and comprising nanoparticles within about 3-5 weeks after application of the first and second dental solutions. The first dental solution comprises an organic or inorganic silver salt and the second dental solution comprises an organic or inorganic reducing agent.

In the foregoing method, at least 40%, or at least 50%, or at least 60%, or at least 70%, or at least 80%, or at least 90%, or at least 95%, or at least 99% of the nanoparticles may have an absolute diameter of 100 nanometers or less, wherein the absolute diameter is measured using high resolution transmission electron microscopy of serial sections prepared from the crust.

In each of the foregoing embodiments, the nanoparticles may comprise silver.

In each of the foregoing embodiments, the first dental solution may comprise silver fluoride; silver fluoride stabilized with nitric acid; silver nitrate; silver diamine fluoride (silver diammine fluoride); silver diammine fluoride stabilized with ammonia; silver diammine nitrate stabilized with ammonia; or a mixture of silver nitrate, boron fluoride, hydrofluoric acid and ammonia hydroxide, and the second dental solution may comprise stannous fluoride.

In each of the foregoing embodiments, the second dental solution may comprise stannous fluoride and the first dental solution may comprise a mixture of silver fluoride and nitric acid.

In each of the foregoing embodiments, the first dental solution may comprise silver fluoride stabilized with nitric acid wherein the nitric acid may be added in sufficient quantity to substantially stabilize the silver fluoride in solution such that the silver ion levels do not decrease by more than 5% over a period of at least 24 months at approximately 25° C.

In each of the foregoing embodiments, the first dental solution may comprise silver fluoride and may be formed in a process comprising dissolving silver fluoride powder in deionized water and removing particulates with a filter to form a filtrate comprising aqueous silver fluoride.

In each of the foregoing embodiments, the deionized water may have a resistivity of 18.2 MΩ·cm, TOC<10 ppb and bacterial count <10 CFU/ml.

In each of the foregoing embodiments, the first dental solution may comprise silver fluoride and may further comprise combining the silver fluoride with nitric acid until a pH of the first dental solution is 4.0-6.0, or 5.5-6.0.

In each of the foregoing embodiments, the first dental solution may comprise silver diamine fluoride wherein the silver diammine fluoride may comprise between 1%-50% w/v silver fluoride.

In each of the foregoing embodiments, the first dental solution may comprise silver diammine fluoride and may be made by dissolving silver fluoride powder in water with the pH adjusted with ammonia between 1%-28% w/v.

In each of the foregoing embodiments, the reducing agent may be stannous fluoride which is formed in a process comprising, step (a) heating anhydrous glycerol to >140° C. to prepare heated glycerol, step (b) adding D-sorbitol powder to the heated glycerol of step (a) while stirring to form a first mixture, step (c) adding powdered stannous fluoride to the first mixture while stirring, and step (d) continue stirring for 20 minutes to 40 minutes until stannous fluoride is dissolved. In step (c), the stannous fluoride may be pulverized. Also, the temperature of step (a) may be at least 170° C., or about 180° C.

In each of the foregoing embodiments, an amount of D-sorbitol may be 1-10 w/v %, or about 5 w/v % in the reducing agent.

In each of the foregoing embodiments, the second dental solution may comprise an amount of stannous fluoride of from 1-50 % w/v, or from 5-25 % w/v, or from about 10 % w/v in the reducing agent (wherein w/v %=mass of solute (g)/vol of soln (ml)×100).

In each of the foregoing embodiments, the first dental solution may comprise between 1%-50% w/v silver fluoride, or 20%-49% w/v silver fluoride, or 30%-45% w/v silver fluoride.

In each of the foregoing embodiments, the inorganic reducing agent may be a metallic salt.

In each of the foregoing embodiments, the inorganic reducing agent may be between 1%-50% w/v stannous fluoride.

In each of the foregoing embodiments, the metallic salt may be at least one of ferrous fluoride or potassium iodide.

In each of the foregoing embodiments, the reducing agent may comprise 1%-50% w/v of the metallic salt.

In each of the foregoing embodiments, the reducing agent may be made without added water, and optionally has added glycerol.

In each of the foregoing embodiments, the reducing agent may comprise up to 100% w/v glycerol in the reducing agent, and between 1% to 20% w/v sorbitol in the reducing agent.

Moreover, in each of the foregoing embodiments, the glycerol may be present in a range of 50 % w/v to 99 % w/v or 70 % w/v to 95% w/v, or 80 % w/v to 93% w/v, based on the total weight of the reducing agent. In addition, in each of the foregoing embodiments, the sorbitol may be present in a range of 0.1 % w/v to 15% w/v, or 0.5 % w/v to 10% w/v, or 1 % w/v to 5 % w/v, based on the total weight of the reducing agent. Preferably, sorbitol is D-sorbitol.

In each of the foregoing embodiments, the reducing agent may be mechanically mixed.

In each of the foregoing embodiments, the organic reducing agent may comprise at least one of tannic acid, a polyphenol, eugenol, a phenylpropanoid, an oligosaccharide, a polysaccharide, a monosaccharide and a disaccharide, a polyethylene glycol, and lactic acid bacteria (Lactobacillus genus).

In each of the foregoing embodiments, the monosaccharide may comprise at least one of aldoses and ketoses classes of organic chemical compounds, and the disaccharide may comprise at least one of lactose, maltose, and calcium sucrose phosphate.

In each of the foregoing embodiments, the monosaccharide may comprise dietary monosaccharides with at least one of galactose, glucose, or fructose.

In each of the foregoing embodiments, the oligosaccharide may comprise at least one of starch or starch derivative, and wherein at least one of starch and starch-derivative may comprise at least one of glucose syrup, maltodextrin, or dextrin.

In each of the foregoing embodiments, the polysaccharide(non-sugars) may comprise at least one of cellulose, starch, glycogen, or chitin.

In each of the foregoing embodiments, the organic reducing agent may be an artificial sweetener/sugar substitute which can be made in the laboratory.

In each of the foregoing embodiments, the sugar substitute/artificial sweetener may be based on at least one of dextrose, maltodextrin, or sucralose.

In each of the foregoing embodiments, the reducing agent may be a flowing solution which can be applied with a microbrush in volumes as low as 0.01 ml and may contain at least one suitable flavouring agent.

In each of the foregoing embodiments, the carious lesion may be in a tooth of a mammal, or wherein the carious lesion may be in a tooth of a human.

Additional details and advantages of the disclosure will be set forth in part in the description which follows, and/or may be learned by practice of the disclosure. The details and advantages of the disclosure may be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

FIG. 1 is a low resolution scanning electron microscopy (SEM) image of the discarded caries surface crust showing relatively evenly spaced holes suggestive of a dentine origin. There is no evidence of bacteria, bacterial remnants, or organic deposits.

FIG. 2 is a series of increasingly higher resolution scanning electron microscopy (SEM) images showing electron-dense particles widely distributed over the discarded crust surface (a) with no obvious clumping (b). The highest resolution view (c) with a 100 nm dot circled shows the majority were this diameter or less.

FIG. 3 shows a photograph in the left image, using energy dispersive spectroscopy for two spot sites selected at random. The circles show the estimated area covered by the electron beam. Both sites have high peak heights for calcium and phosphorus and less for oxygen. Site A has a higher concentration of dense particles than Site B and has more pronounced peak heights for silver, as shown in the middle image and right image, respectively.

FIG. 4 shows an example of a filtration apparatus that can be used in the preparation of the silver fluoride solution.

### DETAILED DESCRIPTION

In an aspect, the invention is drawn to a method of treating an active carious lesion in a tooth comprising steps of: applying a first dental solution to the carious lesion of the tooth, applying a second dental solution to the treated carious lesion within less than 10 minutes of the step of applying the first dental solution, wherein the treated carious lesion consolidates forming a crust having a matte black surface and comprising nanoparticles within about 3-5 weeks after application of the first and second dental solutions. The first dental solution comprises an organic or inorganic silver salt and the second dental solution comprises an organic or inorganic reducing agent. The first dental solution is allowed to diffuse into the active caries surface layer without applying a scrubbing action which would remove carious material containing the diffused first dental solution.

The nanoparticles have an absolute diameter of 100 nanometers or less, wherein the absolute diameter can be measured using scanning electron microscopy or, preferably, transmission electron microscopy and the diameter value used for each particle in the microscopy view is the largest diameter shown in the 2-dimensional view for particles that are spherical or not perfectly spherical. Preferably, at least 40%, or at least 50%, or at least 60%, or at least 70%, or at least 80%, or at least 90%, or at least 95%, or at least 99% of the nanoparticles have an absolute diameter of 100 nanometers or less.

The composition of the nanoparticles depends on the starting materials. The nanoparticles can comprise silver. Without being bound by theory, it is believed that the nanoparticles comprising silver will accelerate the arresting of the carious lesion.

The first dental solution of the present method can comprise aqueous silver fluoride; stabilized with nitric acid; silver nitrate; silver diamine fluoride (a.k.a. silver diammine fluoride); silver diammine fluoride stabilized with ammonia; silver diammine nitrate stabilized with ammonia; or a mixture of silver nitrate, boron fluoride, hydrofluoric acid and ammonia hydroxide. The silver diammine fluoride and silver diammine nitrate can each be stabilized with ammonia in an amount between 1%-28% w/v of the first dental solution. Preferably, the first dental solution is silver fluoride stabilized with nitric acid. The silver fluoride can be combined with nitric acid until a pH of the first dental solution is 4.0-6.0, or 5.5-6.0.

In one or more embodiments, the silver fluoride is formed in a process comprising dissolving silver fluoride in deionized water and removing particulates with a filter to form a filtrate comprising aqueous silver fluoride. The deionized water can have a resistivity of 18.2 MΩ·cm, TOC<10 ppb and bacterial count <10 CFU/ml.

The second dental solution of the present method can comprise an organic or inorganic reducing agent.

The organic reducing agent can have different functional groups such as ketone, aldehyde, hydroxyl, amine(ammonia), and carboxyl groups that can be responsible for the reduction of different metal ions to metal atoms. Aldehyde groups and their concentration in reducing sugars can play a role in nanoparticle formation. The organic reducing agent can comprise at least one of tannic acid, a polyphenol, eugenol, a phenylpropanoid, an oligosaccharide, a polysaccharide, a monosaccharide and a disaccharide, a polyethylene glycol, and lactic acid bacteria (Lactobacillus genus). In one or more embodiments, the monosaccharide can comprise at least one of the aldoses and ketoses classes of organic chemical compounds, and the disaccharide can comprise at least one of lactose, maltose, and calcium sucrose phosphate. Preferably, the monosaccharide can comprise dietary monosaccharides with at least one of galactose, glucose, or fructose. Preferably, the oligosaccharide can comprise at least one of starch or starch derivative comprising glucose syrup, maltodextrin, or dextrin. Preferably, the polysaccharide(non-sugars) can comprise at least one of cellulose, starch, glycogen, or chitin. Preferably, the organic reducing agent can be an artificial sweetener/sugar substitute. The artificial sweetener/sugar substitute can be based on at least one of dextrose, maltodextrin, sucrose, saccharine, sucralose or aspartame or can be natural products such as xylitol and sorbitol. Also, commercially available sugars (white and brown), sugar substitutes and artificial sweeteners brands can be used, such as Splenda™, Sweet'N Low™, Equal Original™, Caribou Coffee™, Great Value™, Stevia™ and Whole Earth™. In one embodiment, the artificial sweetener, such as Splenda™, is used in the absence of Polyvinylpyrrolidone (PVP), a capping agent, to avoid reduced production of silver ions and AgNPs resulting in a high yield of AgNPs. Sugar, sugar substitutes and artificial sweeteners can also be acesulfame, cyclamate, erythritol, and neotame. The sugar, sugar substitutes and artificial sweeteners can have functional groups as NH—, OH, NH₂, OCH₃, or CH₂OH.

The inorganic reducing agent can comprise a metallic salt. The metallic salt can be at least one selected from stannous fluoride, ferrous fluoride or potassium iodide. Preferably, the reducing agent can comprise between 1%-50% w/v of the metallic salt. The reducing agent can be anhydrous, i.e., less than 0.03 % w/v water, or less than 0.005 % w/v water. The reducing agent may have added glycerol. The reducing agent can comprise between 1%-100% w/v glycerol and between 1% to 20% w/v sorbitol. Glycerol can be added at a temperature between 25-185° C. Also, the reducing agent can be a solution which is mechanically mixed. The reducing agent can be a free-flowing solution which can be applied to the carious lesion with a microbrush in volumes as low as 0.01 ml and optionally contains at least one suitable flavoring agent.

Preferably, the inorganic reducing agent can comprise a stannous fluoride composition. The stannous fluoride composition can have no sign of the characteristic white precipitate which identifies deactivated stannous ions. The absence of this white powder precipitate is an accurate visual signal of an active stannous ion which is bioavailable. The stannous fluoride composition can be stable for at least 24 months in high humidity and temperature environments.

The stannous fluoride composition can be formed in a process comprising,
step (a) heating anhydrous glycerol to >140° C. to prepare heated glycerol,
step (b) adding D-sorbitol powder to the heated glycerol of step (a) while stirring to form a first mixture,
step (c) adding powdered stannous fluoride to the first mixture while stirring,
step (d) continue stirring for 20 minutes to 40 minutes until stannous fluoride is dissolved.

Preferably, the process of forming the stannous fluoride composition can comprise pulverizing the stannous fluoride before step (c). Also, the temperature of step (a) can be at least 170° C., or about 180° C. The amount of D-sorbitol can be 1-10 % w/v, or about 5 % w/v. The amount of stannous fluoride can be 1-50% w/v, 5-25 % w/v, or about 10 % w/v in the stannous fluoride composition. The stannous fluoride composition can be a free-flowing liquid. Most preferably, the stannous fluoride composition can have no observed change in stannous ion levels after aging at 55° C., 75% RH for 80 days, (the equivalent of 24 months in a high humidity and temperature environment) i.e., the composition can be a clear solution with no evidence of a white precipitate formed by oxidation of stannous ions.

In one or more embodiments, the second dental solution can comprise a stannous fluoride and the first dental solution can be a mixture of silver fluoride and nitric acid. The nitric acid can be added in sufficient quantity to substantially stabilize the silver fluoride solution such that the silver ion levels do not decrease by more than 5% over a period of at least 24 months at approximately 25° C.

Preferably, the second dental solution can be added within less than 5 minutes of the application of the first dental solution, or the second dental solution can be added within about 3 minutes of the application of the first dental solution. In the mouth, the silver nanoparticles can be formed on the tooth surface within weeks after the application of the stannous fluoride reducing agent.

The black, friable crust formed by the method was seen on the surface of the caries. It had a matte black surface and contained nanoparticles. The crust can have a surface with relatively evenly spaced holes like a dentine framework.

Preferably, the silver nanoparticles act as a reservoir of silver ions, and the reservoir of silver ions continuously replenishes the spent silver ions on the surface. The fact that the reaction occurs in an oral environment at body temperature helps maintain silver ions from the silver nanoparticles at therapeutic levels.

The carious lesion can be in a tooth of a mammal, or wherein the carious lesion can be in a tooth of a human.

Any of the above-described artificial sweetener/sugar substitutes can be used as an additive without the need for it to act as a reducing agent.

### EXAMPLES

The following examples are illustrative, but not limiting of the methods and compositions of the present disclosure.

### Process of Preparing Silver Fluoride

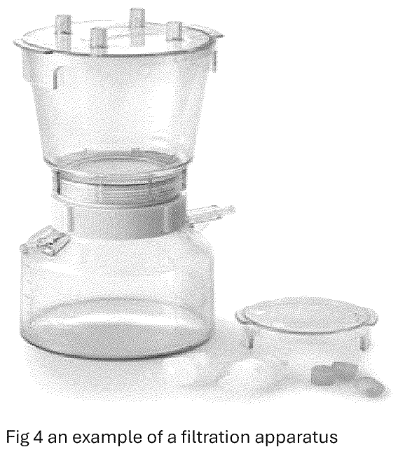

A 40 w/v % silver fluoride solution can be prepared in the following process:
i. Dissolve 40.4 g of 99% pure AgF powder in 90 ml of ultra-pure water in a container. (The amount weighed is equivalent to 40 grams of pure AgF and is calculated as follows: 40×100/99=40.4 g)
ii. Weigh the appropriate filter to be used and place it in an all-plastic filtration apparatus. See FIG. 4 for a suitable example of a filtration apparatus.
iii. Pour contents of the container into the filtration apparatus and apply vacuum.
iv. Once filtration is finished measure the volume of the filtrate. (A)
v. Remove filter and place it in the oven at 110° C. to dry.
vi. Weigh the used filter and subtract from original filter weight to determine the amount of residue from the filtration step.
vii. Subtract this value from the original mass of AgF to determine mass of AgF in the filtrate. (B)
viii. Add 4.0 ml of 70% (w/w) nitric acid. (C).

To determine amount of ultra-pure water (UP water) (D) to be added to make up the final 40% solution, use the following calculation:

$$D = \frac{Mass of AgF in filtrate ( B )}{40} \times 100 minus ( \underline{A} ) + ( \underline{C} )$$

### Process of Preparing Stannous Fluoride

Stannous fluoride can be prepared in the following process:
A) 10.1g 99% pure SnF₂ powder, the amount weighed is calculated as follows: 10×100/99=10.1 g SnF₂ powder.
B) Grind the powder until as fine as possible.
C) Weigh out 5.0 g of D-sorbitol powder.
D) Place 100 ml anhydrous glycerol in a vessel.
E) Heat the 100 ml of glycerol to 180° C.
F) Stir continuously and add 5.0 grams of D-sorbitol powder.
F) Continue to stir until D-sorbitol is completely dissolved.
G) Then add 10.1 g of 99% pure stannous fluoride powder (which is equal to 10 grams pure powder since 10×100/99=10.1 g SnF₂ powder.)
H) Continue to stir for at least 20 minutes (longer for larger volumes) until the stannous fluoride and D-sorbitol are completely dissolved in the glycerol.

### Method of Forming Crust Having a Matte Black Surface and Comprising Nanoparticles

An open active carious lesion on the occlusal surface of a lower primary second molar in a 6-year-old boy was treated with aqueous 40% silver fluoride followed 3 min later by an application of 10% stannous fluoride (Caries Status Disclosing Solution; Creighton Dental). To temporarily exclude saliva, the site was covered with a fluoride varnish.

Three weeks later, when the patient returned to have a glass ionomer cement restoration placed, a black surface crust was seen on the formerly active caries surface. It was scraped off and placed in a 0.2 mL polymerase chain reaction vial with a domed cap and analyzed. No attempt was made to add any form of preservative or transport medium.

The sample was examined using scanning electron microscopy (SEM) and energy dispersive spectroscopy (EDS). This method using SEM is regarded as a valuable tool for the characterization of nanomaterials as it provides a measure of particle size, size distribution, and morphology.

The specimen was dried, mounted, and a conductive coating placed before being examined using SEM with a Zeiss Sigma Field Emission SEM (Carl Zeiss Microscopy) and a Phenom X Desktop SEM (Thermo Scientific). The latter was operated at an acceleration voltage of 15 kV, which would have given an electron beam of just over 1 μm in diameter. Both instruments had energy dispersive spectrum facilities.

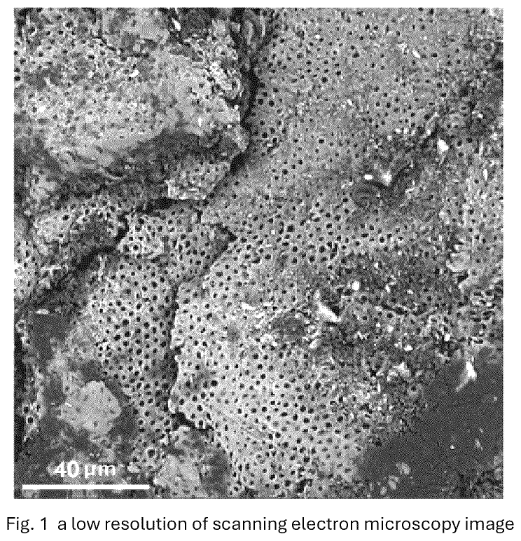

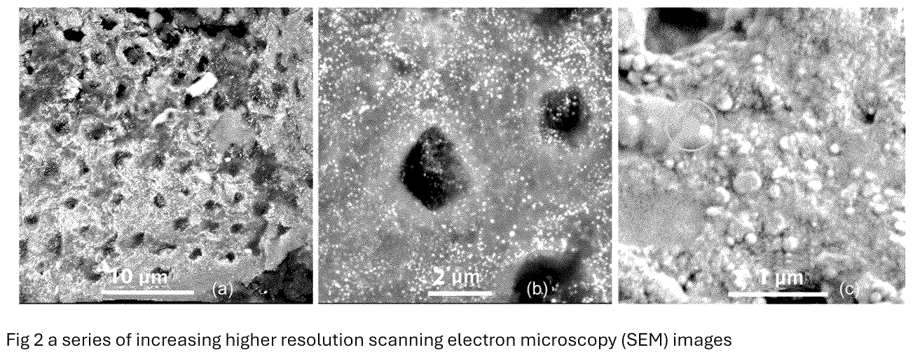

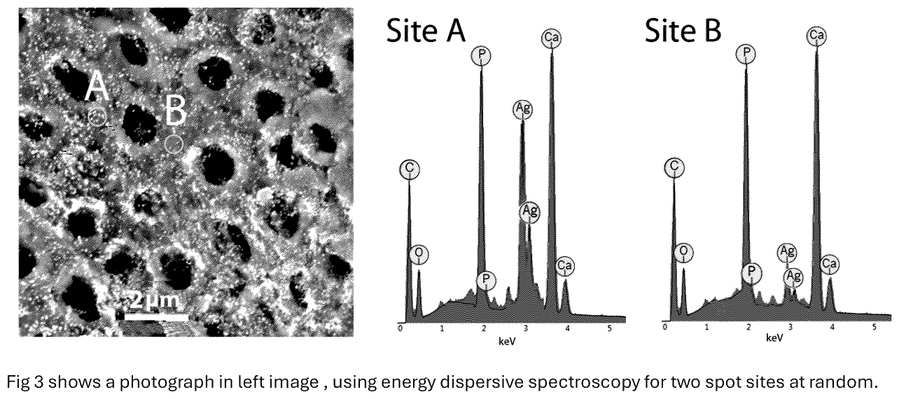

The low-resolution SEM view showed a surface with relatively evenly spaced small holes with an absence of bacteria, bacterial remnants, or organic debris (FIG. 1). Higher resolution views showed small dense particles covering the entire surface, the majority of which were around 100 nm or less in diameter (FIG. 2). Because the surface was not flat or polished and contained porosities it was not feasible to carry out an accurate quantitative EDS analysis. The EDS data for spot analysis of the dense particle sites selected at random all showed marked peak heights for silver as well as calcium, phosphorus, and oxygen. In areas with no obvious dense particles, there were no appreciable peak heights for silver but were present for calcium, phosphorus, and oxygen. Examples of these observations are shown in FIG. 3.

Examination of the removed caries crust gave unexpected findings. The first unexpected finding was that the surface had relatively evenly spaced holes suggestive of a dentine framework. This provided a strong indication that it was derived from dentine beneath the infected surface layer on the original active carious lesion. The absence of any evidence of bacteria or bacterial remnants suggests that the infected layer was lost in the period following the original treatment. Furthermore, the EDS data showed marked peak heights for calcium, phosphorus, and oxygen at all sites examined including ones without any obvious dense particles. This is indicative of some degree of mineralization of the discarded surface crust and whether the elements identified were from the original dentine or picked up from the patient's saliva is open to question. It has been established that SDF can inhibit the degradation of collagen (Mei, Ito, et al., 2013). So it is possible that the silver fluoride in the topical application had a similar effect and helped preserve the dentine architecture.

The second unexpected finding was the observation of the specimen surface coated with small dense particles, the majority of which were 100 nm in diameter or less. EDS findings for sites with these particles gave high peaks for silver. It is noteworthy that in sites where these particles were quite sparse, there were no high peaks for silver. Without being limited to theory, it is believed that the particles are silver nanoparticles.

Several factors could have contributed to the formation of the silver nanoparticles (AgNPs). Initially, the silver ions in the silver fluoride would have been reduced and the stannous ions in the stannous fluoride could perform this function. The stannous ions from stannous chloride have been used in the synthesis of AgNPs (Babaahmadi & Montazer, 2015). Two other constituents of the stannous fluoride solution, glycerol, and sorbitol, could also have played a role. Glycerol is a useful solvent for AgNPs, and sorbitol can help make uniform-sized AgNPs dispersed in a medium. In addition, the bacteria in the carious lesion could have contributed to the bacteria-mediated formation of AgNPs.

Finally, it was unexpected that there was no discernible evidence of either tin or fluoride on the specimen surface even though both the moieties were present in the treatment solutions. Other embodiments of the present disclosure will be apparent to those skilled in the art from consideration of the specification and practice of the embodiments disclosed herein. As used throughout the specification and claims, “a” and/or “an” may refer to one or more than one. Unless otherwise indicated, all numbers expressing quantities of ingredients, properties such as molecular weight, percent, ratio, reaction conditions, and so forth used in the specification and claims are to be understood as being modified in all instances by the term “about,” whether or not the term “about” is present. Accordingly, unless indicated to the contrary, the numerical parameters set forth in the specification and claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. It is intended that the specification and examples be considered as exemplary only, with a true scope and spirit of the disclosure being indicated by the following claims.

The foregoing embodiments are susceptible to considerable variation in practice. Accordingly, the embodiments are not intended to be limited to the specific exemplifications set forth hereinabove. Rather, the foregoing embodiments are within the spirit and scope of the appended claims, including the equivalents thereof available as a matter of law. Other suitable modifications and adaptations of the variety of conditions and parameters normally encountered in the field, and which are obvious to those skilled in the art, are within the scope of the disclosure.

All patents and publications cited herein are fully incorporated by reference herein in their entirety or at least for the portion of their description for which they are specifically cited or relied upon in the present description.

The patentees do not intend to dedicate any disclosed embodiments to the public, and to the extent any disclosed modifications or alterations may not literally fall within the scope of the claims, they are considered to be part hereof under the doctrine of equivalents.

It is to be understood that each component, compound, substituent or parameter disclosed herein is to be interpreted as being disclosed for use alone or in combination with one or more of each and every other component, compound, substituent or parameter disclosed herein.

It is also to be understood that each amount/value or range of amounts/values for each component, compound, substituent or parameter disclosed herein is to be interpreted as also being disclosed in combination with each amount/value or range of amounts/values disclosed for any other component(s), compounds(s), substituent(s) or parameter(s) disclosed herein and that any combination of amounts/values or ranges of amounts/values for two or more component(s), compounds(s), substituent(s) or parameters disclosed herein are thus also disclosed in combination with each other for the purposes of this description.

It is further understood that each range disclosed herein is to be interpreted as a disclosure of each specific value within the disclosed range that has the same number of significant digits. Thus, a range of from 1-4 is to be interpreted as an express disclosure of the values 1, 2, 3 and 4.

It is further understood that each lower limit of each range disclosed herein is to be interpreted as disclosed in combination with each upper limit of each range and each specific value within each range disclosed herein for the same component, compounds, substituent or parameter. Thus, this disclosure to be interpreted as a disclosure of all ranges derived by combining each lower limit of each range with each upper limit of each range or with each specific value within each range, or by combining each upper limit of each range with each specific value within each range.

Furthermore, specific amounts/values of a component, compound, substituent or parameter disclosed in the description or an example is to be interpreted as a disclosure of either a lower or an upper limit of a range and thus can be combined with any other lower or upper limit of a range or specific amount/value for the same component, compound, substituent or parameter disclosed elsewhere in the application to form a range for that component, compound, substituent or parameter.

### Industrial Applicability

This process may be scaled to any commercial quantity required, and is applicable to manufacturers of consumable dental materials.

### References

Babaahmadi, V., & Montazer, M. (2015). A new route to synthesis silver nanoparticles on polyamide fabric using stannous chloride. The Journal of the Textile Institute, 106, 970-977.
Craig, G. G., Powell, K. R., & Price, C. A. (2013). Clinical evaluation of a modified silver fluoride application technique designed to facilitate lesion assessment in outreach programs. BMC Oral Health, 13, 73.
Klanliang, K., Asahi, Y., Maezono, H., Sotozono, M., Kuriki, N., Machi, H., Ebisu, S., & Hayashi, M. (2022). An extensive description of the microbiological effects of silver diamine fluoride on dental biofilms using an oral in situ model. Scientific Reports, 12(1), 7435.
Klaus, T., Joerger, R., Olsson, E., & Granqvist, C. G. (1999). Silver-based crystalline nanoparticles, microbially fabricated. Proceedings of the National Academy of Sciences of the United States of America, 96, 13611-13614.
Lin, P. C., Lin, S., Wang, P. C., & Sridhar, R. (2014). Techniques for physicochemical characterization of nanomaterials. Biotechnology Advances, 32, 711-726.
Liu, T., Baek, D. R., Kim, J. S., Joo, S. W., & Lim, J. K. (2020). Green synthesis of silver nanoparticles with size distribution depending on reducing species in glycerol at ambient pH and temperatures. ACS Omega, 5, 16246-16254.
Mei, M. L., Chu, C. H., Low, K. H., Che, C. M., & Lo, E. C. (2013). Caries arresting effect of silver diamine fluoride on dentine carious lesion with S. mutans and L. acidophilus dual-species cariogenic biofilm.
Medicina Oral, Patología Oral y Cirugía Bucal, 18, e824-e831. Mei, M. L., Ito, L., Cao, Y., Li, Q.L., Lo, E. C., & Chu, C. H. (2013). Inhibitory effect of silver diamine fluoride on dentine demineralisation and collagen degradation. Journal of Dentistry, 41, 809-817.
Mei, M. L., Ito, L., Cao, Y., Lo, E. C. M., Li, Q. L., & Chu, C. H. (2014). An ex vivo study of arrested primary teeth caries with silver diamine fluoride therapy. Journal of Dentistry, 42, 395-402.
Natarajan, D. (2022). Silver Modified Atraumatic Restorative Technique: A way towards “SMART” pediatric dentistry during the COVID-19 pandemic. Frontiers in Dentistry, 19, 12.
Steinigeweg, D., & Schlücker, S. (2012). Monodispersity and size control in the synthesis of 20-100 nm quasi-spherical silver nanoparticles by citrate and ascorbic acid reduction in glycerol-water mixtures. Chemical Communications, 48, 8682-8684.

1. A method of treating an active carious lesion in a tooth comprising steps of:
applying a first dental solution to the carious lesion of the tooth,
applying a second dental solution to the treated carious lesion within less than 10 minutes of the step of applying the first dental solution,
wherein the treated carious lesion consolidates forming a crust having a matte black surface and comprising silver nanoparticles within about 3-5 weeks after application of the first and second dental solutions, and
wherein the first dental solution comprises an organic or inorganic silver salt and the second dental solution comprises an organic or inorganic reducing agent.

2. The method of claim 1, wherein at least 40%, or at least 50%, or at least 60%, or at least 70%, or at least 80%, or at least 90%, or at least 95%, or at least 99% of the nanoparticles have an absolute diameter of 100 nanometers or less, wherein the absolute diameter is measured using high resolution transmission electron microscopy of serial sections prepared from the crust.

3. The method of any one of the previous claims, wherein the nanoparticles comprise silver.

4. The method of any one of the previous claims, wherein
the first dental solution comprises silver fluoride; silver fluoride stabilized with nitric acid; silver nitrate; silver diamine fluoride (silver diammine fluoride); silver diammine fluoride stabilized with ammonia; silver diammine nitrate stabilized with ammonia; or a mixture of silver nitrate, boron fluoride, hydrofluoric acid and ammonia hydroxide, and
the second dental solution comprises stannous fluoride.

5. The method of any one of the previous claims, wherein the second dental solution comprises stannous fluoride and the first dental solution comprises a mixture of silver fluoride and nitric acid.

6. The method of any one of the previous claims, wherein the first dental solution comprises silver fluoride stabilized with nitric acid and the nitric acid is added in sufficient quantity to substantially stabilize the silver fluoride in solution such that the silver ion levels do not decrease by more than 5% over a period of at least 24 months at approximately 25° C.

7. The method of any one of the previous claims, wherein the first dental solution comprises silver fluoride and is formed in a process comprising dissolving silver fluoride powder in deionized water and removing particulates with a filter to form a filtrate comprising aqueous silver fluoride.

8. The method of any one of the previous claims, wherein the deionized water has a resistivity of 18.2 MΩ·cm, TOC<10 ppb and bacterial count <10 CFU/ml.

9. The method of any one of the previous claims, wherein the first dental solution comprises silver fluoride and further comprises combining the silver fluoride with nitric acid until a pH of the first dental solution is 4.0-6.0, or 5.5-6.0.

10. The method of any one of the previous claims, wherein the first dental solution comprises silver diamine fluoride wherein the silver diammine fluoride comprises between 1%-50% w/v silver fluoride.

11. The method of any one of the previous claims, wherein the first dental solution comprises silver diammine fluoride and is made by dissolving silver fluoride powder in water with the pH adjusted with ammonia between 1%-28% w/v.

12. The method of any one of the previous claims, wherein the reducing agent is stannous fluoride, which is formed in a process comprising,
step (a) heating anhydrous glycerol to >140° C. to prepare heated glycerol,
step (b) adding D-sorbitol powder to the heated glycerol of step (a) while stirring to form a first mixture,
step (c) adding powdered stannous fluoride to the first mixture while stirring, and
step (d) continue stirring for 20 minutes to 40 minutes until stannous fluoride is dissolved.

13. The method of claim 12, further comprising pulverizing the stannous fluoride before step (c).

14. The method of any one of claims 12-13, wherein the temperature of step (a) is at least 170° C., or about 180° C.

15. The method of any one of claims 12-14, wherein an amount of D-sorbitol is 1-10 w/v %, or about 5 w/v % in the reducing agent.

16. The method of any one of the previous claims, wherein the second dental solution comprises an amount of stannous fluoride of from 1-50 w/v %, or from 5-25 w/v %, or from about 10 w/v % in the reducing agent.

17. The method of any one of the previous claims, wherein the first dental solution comprises between 1%-50% w/v silver fluoride, or 20%-49% w/v silver fluoride, or 30%-45% w/v silver fluoride.

18. The method of any one of the previous claims, wherein the inorganic reducing agent is a metallic salt.

19. The method of any one of the previous claims, wherein the inorganic reducing agent is between 1%-50% w/v stannous fluoride.

20. The method of any one of the previous claims, wherein the metallic salt is at least one of ferrous fluoride or potassium iodide.

21. The method of any one of the previous claims, wherein the reducing agent comprises 1%-50% w/v of the metallic salt.

22. The method of any one of the previous claims, wherein the reducing agent is made without added water, and optionally has added glycerol.

23. The method of any one of the previous claims, wherein the reducing agent comprises up to 100% w/v glycerol in the reducing agent, and between 1% to 20% w/v sorbitol in the reducing agent.

24. The method of any one of the previous claims, wherein the reducing agent is mechanically mixed.

25. The method of any one of the previous claims, wherein the organic reducing agent comprises at least one of tannic acid, a polyphenol, eugenol, a phenylpropanoid, an oligosaccharide, a polysaccharide, a monosaccharide and a disaccharide, a polyethylene glycol, and lactic acid bacteria (Lactobacillus genus).

26. The method of claim 25, wherein the monosaccharide comprises at least one of aldoses and ketoses classes of organic chemical compounds, and the disaccharide comprises at least one of lactose, maltose, and calcium sucrose phosphate.

27. The method of any one of claims 25-26, wherein the monosaccharide comprises dietary monosaccharides with at least one of galactose, glucose, or fructose.

28. The method of any one of claims 25-27, wherein the oligosaccharide comprises at least one of starch or starch derivative, and wherein at least one of starch and starch-derivative comprise at least one of glucose syrup, maltodextrin, or dextrin.

29. The method of any one of claims 25-28, wherein the polysaccharide(non-sugars) comprises at least one of cellulose, starch, glycogen, or chitin.

30. The method of any one of the previous claims, wherein the organic reducing agent is an artificial sweetener/sugar substitute which can be made in the laboratory.

31. The method of claim 30, wherein the sugar substitute/artificial sweetener is based on at least one of dextrose, maltodextrin, or sucralose.

32. The method of any one of the previous claims, wherein the reducing agent is a flowing solution which can be applied with a microbrush in volumes as low as 0.01 ml and contains at least one suitable flavouring agent.

33. The method of any one of the previous claims, wherein the carious lesion is in a tooth of a mammal, or wherein the carious lesion is in a tooth of a human.
